(19) United States
(12) Patent Application Publication
(10) Pub. No.: US 2011/0040262 A1
(43) Pub. Date: Feb. 17, 2011

(54) DEVICE AND METHODS TO FACILITATE CANNULATION AND PREVENT NEEDLE PUNCTURE BLEEDING OF AN ARTERIOVENOUS FISTULA

(76) Inventor: David L. Cull, Greenville, SC (US)

Correspondence Address: DORITY & MANNING, P.A., POST OFFICE BOX 1449, GREENVILLE, SC, 29602-1449, US

(21) Appl. No.: 12/681,731

(22) PCT Filed: Oct. 6, 2008
(86) PCT No.: PCT/US08/78912

§ 371 Date: Nov. 3, 2010

Related U.S. Application Data
(60) Provisional application No. 60/977,486, filed on Oct. 4, 2007.

(51) Int. Cl. A61M 5/32

(52) U.S. Cl. 604/272

(57) ABSTRACT

In general, the present application is directed to devices and methods to facilitate cannulation and prevent needle puncture bleeding of an arteriovenous fistula. For instance, in one embodiment, a device for locating an arteriovenous fistula and occluding an arteriovenous fistula needle puncture site is provided. The device includes a body formed from a self-sealing biocompatible material, the body having a semi-curved shape with two edges that extend along the length of the body. The body is configured to conform to the contour of a portion of an arteriovenous fistula with the edges configured to be generally parallel to the length of an arteriovenous fistula. The edges provide a tactile clue when focal pressure is applied to the skin above an arteriovenous fistula. The body provides a barrier to limit bleeding from a posterior portion of an arteriovenous fistula.

### CROSS REFERENCE TO RELATED APPLICATION

The present application is based on and claims priority to U.S. Provisional Application Ser. No. 60/977,486 having a filing date of Oct. 4, 2008.

### BACKGROUND

End-stage renal disease (ESRD) is characterized by a complete or near complete failure of the kidneys to function to excrete wastes, concentrate urine, and regulate electrolytes. In such cases, kidney function is so low that complications are multiple and severe, and death will occur from accumulation of fluids and waste products in the body.

A common life-sustaining treatment for patients with ESRD is hemodialysis. Hemodialysis is a process whereby large amounts of blood are rapidly removed from the body and filtered through a machine that removes wastes and extra fluid. The cleaned blood is then returned back into the body.

An important step before starting regular hemodialysis is preparing a vascular access, which is a site on the body where blood will be removed and returned during dialysis. In this regard, creation of an arteriovenous fistula (AV fistula) is a commonly performed operation in which an artery is connected directly to a vein. The high blood pressure of the artery causes more blood to flow into the vein and, as a result, the vein dilates growing larger and stronger.

However, to connect the patient to a dialysis machine, a nurse or some other medical technician must insert a large gauge needle through the skin into the AV fistula. The technique of cannulating an AV fistula for dialysis requires considerable skill. The AV fistula often lies several centimeters below the surface of the skin and cannot be located by visual inspection. A medical technician is forced to locate the AV fistula by palpation. Since resistance to blood flow in the vein is low, a pulse is usually not present in the AV fistula. The tactile clue utilized to locate the AV fistula is a vibration caused by turbulent blood flow in the vein. The medical technician tries to identify the location of maximum vibration on the surface of the skin with his/her fingertip to identify the location of the underlying AV fistula in order to cannulate it.

If the medical technician is unable to properly identify the correct location of the AV fistula, the dialysis needle may inadvertently puncture the side rather than the center of the AV fistula and result in damage and significant bleeding or thrombosis of the AV fistula. Damage and significant bleeding or thrombosis can also occur if the dialysis needle passes through the back wall of the AV fistula. While direct pressure can be applied to a fistula needle puncture site that is adjacent to the skin, puncture to other areas of a fistula are of greater concern because direct pressure cannot be applied without occluding the fistula.

Currently, vascular grafts exist which can bypass blood flow from one point on a blood vessel to a different point on the vessel or to a different blood vessel entirely. In the field of vascular access surgery, a technique has been described whereby a surgeon splits such a vascular graft longitudinally and wraps the graft circumferentially around an AV fistula. The purpose of this technique is to allow a technician to more easily identify the AV fistula beneath the skin because the graft is more easily palpated than the AV fistula.

Unfortunately, the infection rate of arteriovenous grafts when compared to AV fistulas is significantly higher because during cannulation, bacteria on the skin surface are “inoculated” into the graft material by the dialysis needle. Once this occurs, bacteria are resistant to immune defenses which can fight infection as well as to antibiotics which treat infection. By comparison, bacteria that are inoculated into AV fistulas are susceptible to immune defenses of the body and infections in AV fistulas are rare.

In order to cannulate an AV fistula with a needle using the circumferential graft technique described above, the needle must pass through the graft. As such, a fundamental advantage of an AV fistula over the graft is lost when utilizing the circumferential graft technique. Indeed, the infection rate of the circumferential graft technique is likely identical to that of a standard arteriovenous graft. Furthermore, there is no discussion of the circumferential graft technique being utilized to prevent bleeding from inadvertent punctures of an AV fistula.

Thus, a need exists for a device and method that can simplify the technique of AV fistula cannulation. Furthermore, a need exists for a device that will prevent bleeding from an AV fistula caused by a dialysis needle that inadvertently punctures an area of an AV fistula that is not adjacent to the skin.

### SUMMARY

In general, the present application is directed to devices and methods to facilitate cannulation and prevent needle puncture bleeding of an arteriovenous fistula. For instance, in one embodiment, a device for locating an arteriovenous fistula and occluding an arteriovenous fistula needle puncture site is provided. The device includes a body formed from a self-sealing biocompatible material, the body having a semi-curved shape with two edges that extend along the length of the body. The body is configured to conform to the contour of a portion of an arteriovenous fistula with the edges configured to be generally parallel to the length of an arteriovenous fistula. The edges provide a tactile clue when focal pressure is applied to the skin above an arteriovenous fistula. The body provides a barrier to limit bleeding from a posterior portion of an arteriovenous fistula.

In still another embodiment, a method for locating an arteriovenous fistula and occluding an arteriovenous fistula needle puncture site is provided. The method includes providing a device having a body, the body formed from a self-sealing biocompatible material. The body has a semi-curved shape with two edges that extend along the length of the body. The device is positioned so that the body conforms to the contour of a portion of an arteriovenous fistula with the edges generally parallel to the length of the arteriovenous fistula. The body provides a barrier to limit bleeding from the posterior wall of the arteriovenous fistula. A tactile clue is detected from the edges of the body when focal pressure is applied to the skin above the arteriovenous fistula.

Other features and aspects of the present invention are discussed in greater detail below.

### DESCRIPTION OF THE DRAWINGS

A full and enabling disclosure, including the best mode thereof to one of ordinary skill in the art, is set forth more particularly in the remainder of the specification, including reference to the accompanying figures in which:

FIG. 1 depicts an AV fistula in accordance with one embodiment of the present disclosure;

FIGS. 2A-D illustrate successive steps of detecting and cannulating an AV fistula;

FIGS. 3A-B depict a device positioned along a segment of an AV fistula in accordance with one embodiment of the present disclosure;

FIGS. 4A-C depict a device positioned along a segment of an AV fistula in accordance with one embodiment of the present disclosure; and

FIG. 5 depicts a tunneler for implanting a device in accordance with the present disclosure; and

FIGS. 6A-D depict a device being implanted in accordance with one embodiment of the present disclosure.

### DETAILED DESCRIPTION

It is to be understood by one of ordinary skill in the art that the present discussion is a description of exemplary embodiments only, and is not intended as limiting the broader aspects of the present invention, which broader aspects are embodied in the exemplary construction.

In general, the present disclosure is directed to devices and methods for locating an arteriovenous (AV) fistula and occluding an AV fistula needle puncture site. In particular, the devices and methods described herein greatly simplify the technique of arteriovenous fistula cannulation by improving a technician's ability to locate an AV fistula and occlude unintentional needle punctures to the AV fistula. The devices and methods described herein can help to occlude needle puncture sites until hemostasis is achieved while avoiding potential thrombosis of the AV fistula.

Very generally, a fistula is a connection between two parts of the body that are usually separate. An AV fistula is useful because it allows for easy access to the blood system of a patient. Creation of an AV fistula is a commonly performed operation in which an artery is connected directly to a vein. The high blood pressure of the artery causes more blood to flow into the vein and, as a result, the vein dilates growing larger and stronger. An AV fistula is considered the best long-term vascular access for hemodialysis because it provides adequate blood flow for dialysis, lasts a long time, and has a complication rate lower than other types of access. When cannulated correctly, a properly formed AV fistula is less likely than other kinds of vascular accesses to form clots or become infected. Also, AV fistulas tend to last many years, longer than any other kind of vascular access.

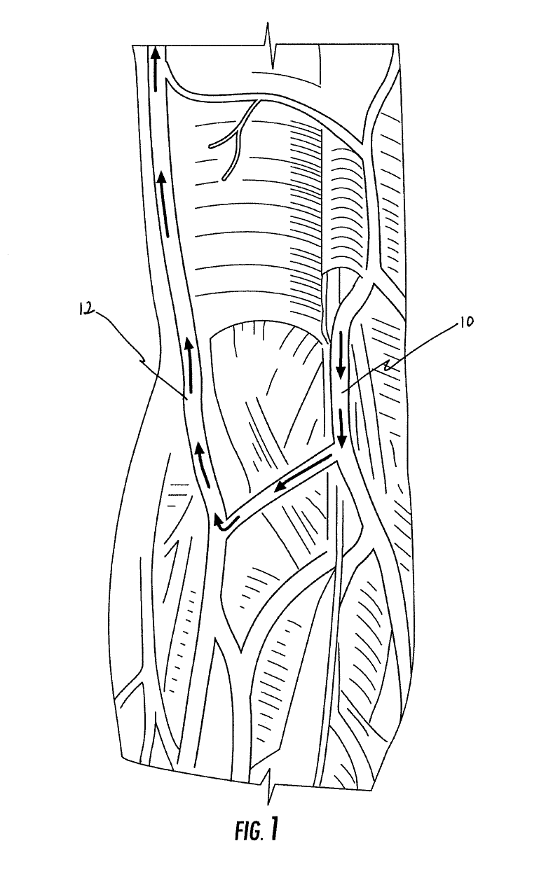

Any suitable AV fistula is contemplated for use in connection with the present disclosure. For instance, referring to FIG. 1, a brachiocephalic fistula is illustrated. In a brachiocephalic fistula, the brachial artery 10 is connected to the cephalic vein 12. However, any suitable AV fistula can be utilized. In addition, when suitable superficial veins are not available for AV fistula construction, there are often deeper veins that are more suitable. These veins and their suitability can be identified by vessel mapping and then surgically repositioned (transposed) to a superficial location suitable for cannulation. Transposition can also be done for veins that may be superficial enough but not positioned for safe cannulation. The present disclosure specifically contemplates use with such transposed veins.

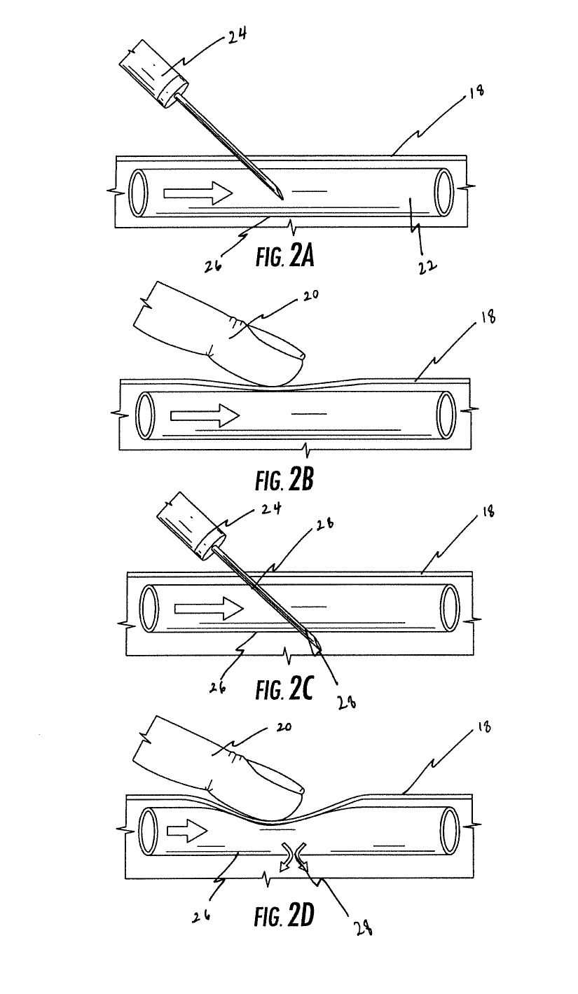

Once an AV fistula is formed, the tactile clue that a technician uses to locate it (a vibration caused by turbulent blood flow in the vein) is difficult to locate at best. Referring to FIGS. 2A-D, a technician tries to identify the location of maximum vibration on the surface of the skin 18 with his/her fingertip 20 to identify the location of the underlying AV fistula 22 in order to cannulate it. If the dialysis needle 24 inadvertently punctures the side rather than the center of the AV fistula 22, damage and significant bleeding or thrombosis of the AV fistula 22 can result. Similarly, damage and significant bleeding or thrombosis can also occur if the dialysis needle 24 punctures the back wall of the AV fistula 22. While direct pressure can be applied to a fistula needle puncture site 28 that is adjacent to the skin 18 to stop bleeding, puncture to posterior areas 26 of an AV fistula are of greater concern because direct pressure cannot be applied without potentially occluding the AV fistula. Indeed, referring to FIG. 2D, direct pressure can actually result in increased bleeding to the posterior areas 26 of an AV fistula.

The present devices and methods seek to eliminate such a negative outcome by enhancing a technician's ability to locate an AV fistula through an improved tactile clue to locate the AV fistula. The devices and methods can also elevate an AV fistula, further assisting a technician in locating the AV fistula for cannulation while potentially reducing the incidence of tortuous fistulas. In addition, the present devices and methods assist in occluding posterior needle puncture sites, should they occur. An AV fistula can also be prevented from rolling by the present devices and methods, which can be a major cause of missed needle sticks.

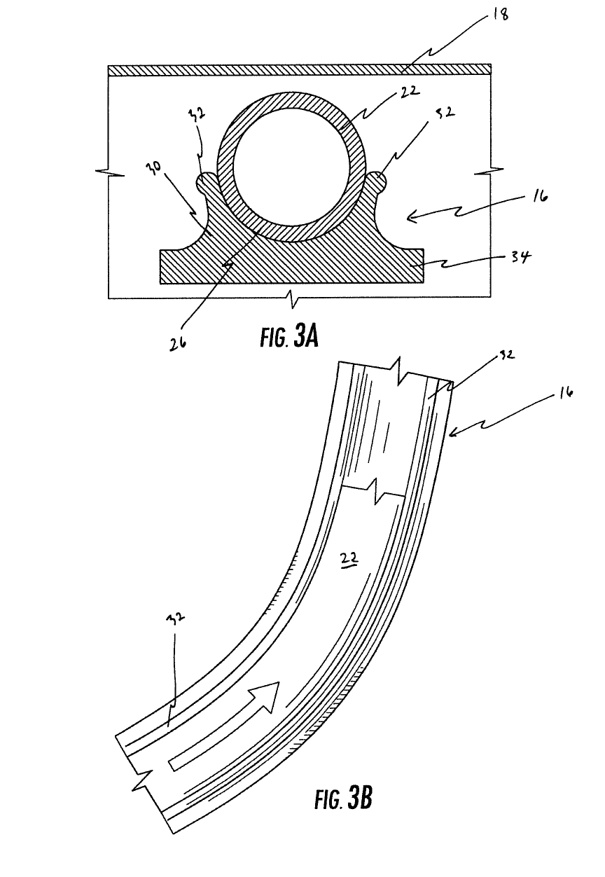

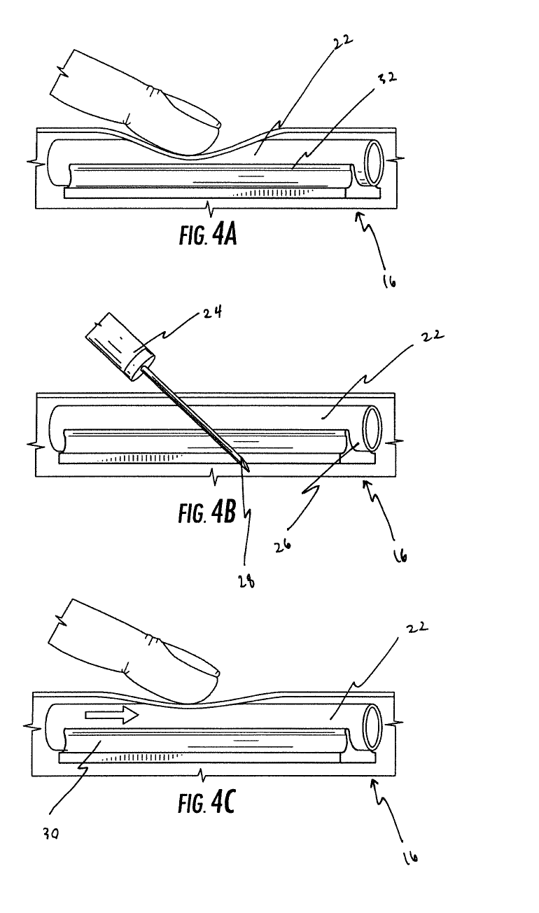

In that regard, the present disclosure relates to an implantable device for locating an AV fistula and occluding an AV fistula needle puncture site. With reference to FIGS. 3A and 3B, the device 16 includes a body 30 having two edges 32 and a base 34.

The body 30 has a semi-curved shape which can conform to the contour of an AV fistula 22 so as to be positioned along the posterior portion 26 of the AV fistula 22. As used herein, the posterior portion of an AV fistula refers to any portion of the AV fistula that is not located directly beneath the surface of the skin so as to prevent the application of direct pressure when occluding the AV fistula. In this manner, the body 30 can cradle an AV fistula 22. The body 30 can have a depth of from about 2 mm to about 8 mm and a width of from about 5 mm to about 20 mm.

The body 30 can be formed from any suitable self-sealing biocompatible material. In this manner, the body 30 provides a barrier to limit bleeding from a posterior portion of an AV fistula. Without limitation, suitable materials utilized to form the body can include polymers and copolymers, including thermoplastic elastomers and certain silicones, silicone rubbers, synthetic rubbers, polyurethanes, polyethers, polyesters, polyamides, various flouropolymers, including, but not limited to polytetrafluroethylene, expanded polytetrafluroethylene, and mixtures thereof.

The body 30 can be self-sealing or can be impregnated with a gel to provide self-sealing capabilities or improved self-sealing capabilities. Examples of such gels include hydrogels formed from natural materials including, but not limited to, gelatin, collagen, albumin, casein, algin, carboxy methyl cellulose, carageenan, furcellaran, agarose, guar, locust bean gum, gum arabic, hydroxyethyl cellulose, hydroxypropyl cellulose, methyl cellulose, hydroxyalkylmethyl cellulose, pectin, partially deacetylated chitosan, starch and starch derivatives, including amylose and amylopectin, xanthan, polylysine, hyaluronic acid, and its derivatives, heparin, their salts, and mixtures thereof.

Additionally, the body 30 can include therapeutic agents which can aid in healing an AV fistula needle puncture site or the surrounding tissue. The therapeutic agent can be located on or in the body 30 (including incorporated into the material forming the body 30) in many forms including but not limited to fluids, gels, solids, suspensions, emulsions, slow-release or time-release beads/microsphere, nanoparticles, capsules, liposomes, cells, tissue, ion-exchange beads, biodegradable polymers, pellets, or other micro/nano-particulate forms.

The body 30 includes two edges 32. The edges 32 provide a tactile clue when focal pressure is applied to surface of the skin 18 above an AV fistula 22. The edges 32 are formed so as to have features that make them easily palpable on the skin surface 18 even though the device is implanted beneath the surface. Such features can include ridges, bumps, or any other feature that would be suitable to allow the edges 32 to be palpable on the skin surface 18. The distance between the two edges 32 of body 30 can be from about 8 mm to about 16 mm. The edges 32 can be integrally connected with the body 30 or can be joined to the body 30 by some other method. If the edges 32 are joined to the body, any suitable method as would be known in the art can be utilized including adhesives, fasteners, and the like.

As indicated above, the body 30 can include a base 34. As illustrated, the base 34 can generally be wider than the distance between the edges of body 30 so as to provide a stable base and stabilize a fistula placed in body 30. The base can have a width of from about 5 mm to about 20 mm and more particularly from about 8 mm to about 18 mm. In certain embodiments, the base can have a width of about 12 mm. The base can have a generally flat bottom surface and can have a height that serves to elevate the body. When implanted, the height can help make a fistula located in body easier to find for cannulation. The sides of the base can be generally flat and can each extend to a curved surface that joins the body. However, it is contemplated that the base can have any size and/or shape as would be appreciated in the art. The base 34 can be integrally connected with the body 30 or can be joined to the body 30 by some other method. If the base 34 is joined to the body, any suitable method as would be known in the art can be utilized including adhesives, fasteners, and the like.

In certain embodiments, the body 30 can include a reinforcing material (not illustrated). The reinforcing material can be of any form suitable to reinforce the body 30 so as to keep the edges 32 upright. For instance, in some embodiments, the reinforcing material can be a wire coil surrounding the body 30. However, it should be understood that any suitable reinforcing material can be utilized. The reinforcing material can be formed from any suitable biocompatible material including metals such as stainless steel or nitinol, or a structurally suitable polymer material.

The device 16 of the present disclosure can be of any suitable size. In particular, the length and width of the device can correspond to the length and width of the AV fistula that the device is used in connection with. In certain embodiments, the device can have a length of from about 20 cm to about 50 cm, and a width of from about 0.5 cm to about 2 cm. However, it should be understood that any suitable length or width is contemplated by the present disclosure.

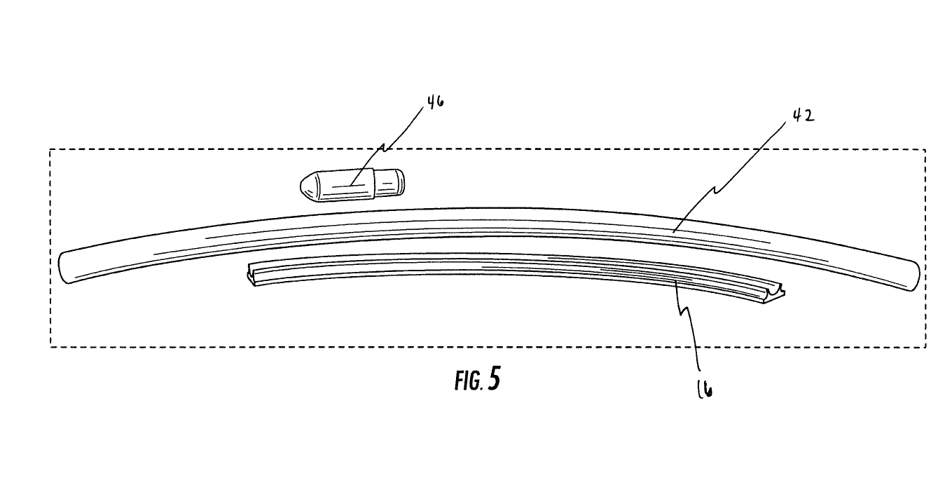

Referring again to FIGS. 4A-C, use of the device 16 of the present disclosure will be described in detail. As illustrated in FIG. 4A, prior to cannulation, a technician is able to locate an AV fistula 22 through a tactile clue from the edges 32 of the device 16. Once a technician locates the AV fistula 22, he/she can cannulate the AV fistula 22 by inserting the dialysis needle 24 between the edges 32 of the device 16 into the AV fistula 22.

In the event that the dialysis needle 24 passes through a posterior area 26 of the AV fistula 22, the device 16 of the present disclosure provides a resealable barrier. Referring to FIG. 4B, a depiction of a needle 24 puncturing an AV fistula 22 to form a posterior puncture site 28 is illustrated. As shown in FIG. 4C, once the needle 24 is withdrawn, the self-sealing body 30 of the device 16 is capable of occluding the needle puncture site 28 of the AV fistula 22 to prevent internal bleeding.

The device 16 of the present disclosure can be implanted by any suitable method as would be known in the art. In particular, the device 16 can be implanted during a procedure to create an AV fistula or to revise an AV fistula. The device 16 is positioned by a surgeon along the outside, posterior wall of the AV fistula, thereby cradling the AV fistula. The device 16 should be positioned along the portion of the fistula which is to be cannulated. As described above, the length and width of the device can be adjusted to best accommodate the anatomic differences between patients. In that regard, the device 16 can be sized and trimmed, if necessary, during the procedure to implant the device 16.

After implantation, the device becomes fused to an AV fistula through a normal healing process known as incorporation. Specifically, the posterior wall of an AV fistula will become fused to the inner surface of the body of the device 16 and the outer surface of the device will become fused to surrounding subcutaneous tissue. For example, in certain embodiments, surrounding subcutaneous tissue can grow over base 33 and securely attach device 16 such that a stable base is provided for an AV fistula.

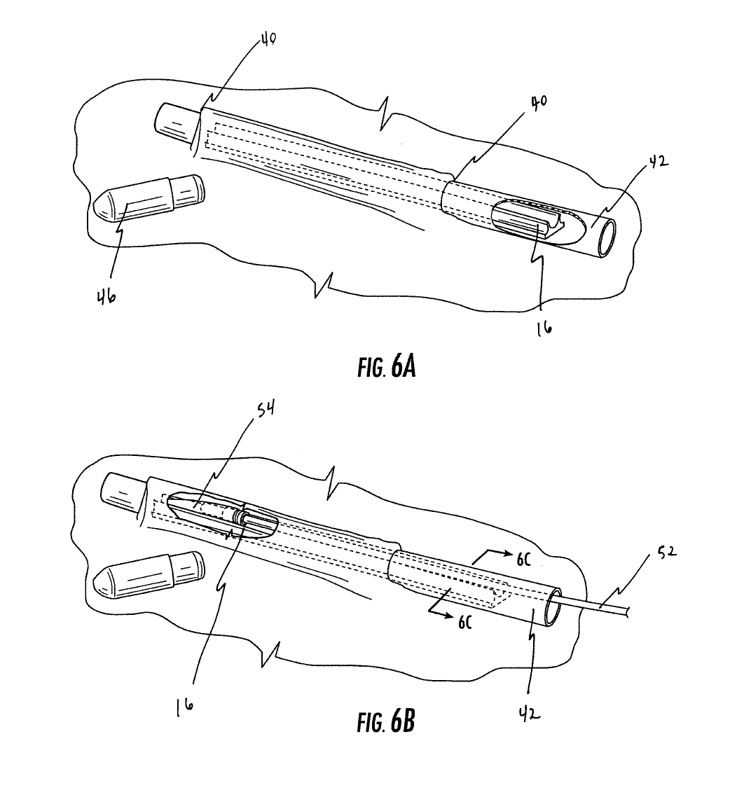

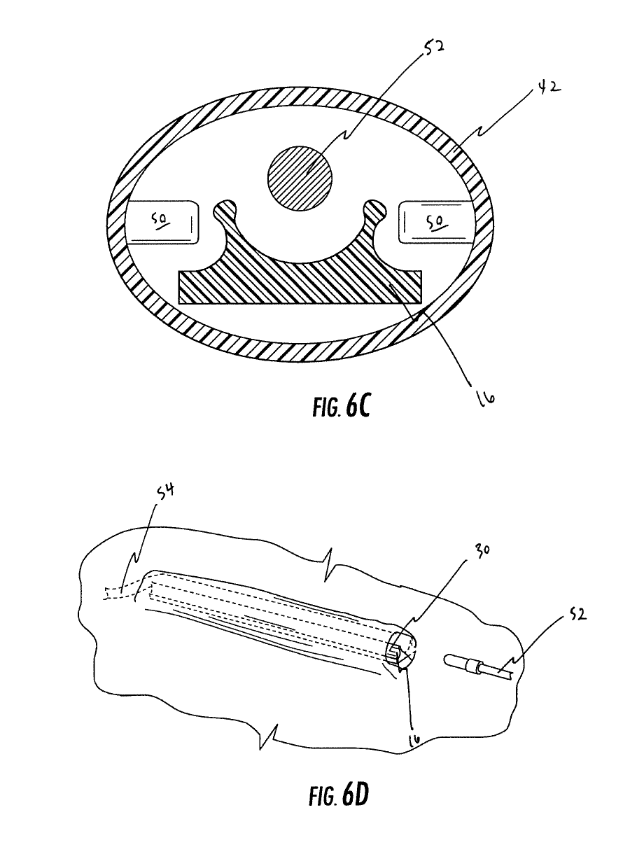

Turning to FIG. 5, a method for implanting a device 16 of the present disclosure is illustrated. A tunneler 42 is provided and the device 16 is placed inside. Tunneler 42 includes a tube 44 and a cap 46. Cap 46 removably attaches at an end of tube 44 and can have a generally pointed surface so as to allow the tunneler 42 to be more easily inserted underneath the skin. Device 16 is placed in the interior cavity 48 of tunneler 42. For instance, the interior cavity 48 of the tunneler 42 can include one or more guide members 50 (illustrated in FIG. 6C). The guide members can assist in orienting the device 16 with the base 33 at the bottom of the tunneler 42.

The tunneler can be formed from any suitable biocompatible material including a semi-rigid plastic material or the like. In this regard, any suitable tunneler as would be known in the art can be utilized. For instance, SCANLAN® tunnelers are useful in connection with the present disclosure. Again, however, any suitable tunneler is contemplated for use in the present disclosure.

As shown in FIG. 6A, two incisions 40 are made in the skin at the site of the implantation. Tunneler 42 and device 16 contained therein are inserted through one incision and exited through the other incision. Cap 46 is removed from tunneler 42 exposing device 16. Referring to FIG. 6B, a tunneling rod 52 is inserted through the length of tunneler 42 with the rod being positioned above device 16 and exiting from the end of tunneler 42 opposite from that into which it was inserted. A vein 54 is joined to rod 52. The vein 54 can be tied to rod 52 or attached in any other suitable manner as would be known in the art. As shown in FIG. 6D, rod 52 is pulled back through tunneler 42 until it is completely removed, thereby resulting in vein 54 being pulled through tunneler 42 as well. The vein 54 is disconnected from rod 52 and the tunneler 42 is carefully removed from the incision site leaving the device 16 implanted with the vein 54 resting in body 30 of device 16. The end of vein 54 can then be joined to an artery and the incisions can be sewn closed. In this manner, the device of the present disclosure can be implanted with an AV fistula positioned thereon such that the posterior wall of the AV fistula is cradled by the device.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed is:

1. A device for locating an arteriovenous fistula and occluding an arteriovenous fistula needle puncture site, the device comprising:
a body, the body formed from a self-sealing biocompatible material, the body having a semi-curved shape with two edges that extend along the length of the body, the body configured to conform to the contour of a portion of an arteriovenous fistula with the edges configured to be generally parallel to the length of an arteriovenous fistula, the edges providing a tactile clue when focal pressure is applied to the skin above an arteriovenous fistula, the body providing a barrier to limit bleeding from a posterior portion of an arteriovenous fistula.

2. The device of claim 1, wherein the body further comprises a base, the base having a width that is greater than the distance between the two edges of the body.

3. The device of claim 2, wherein the base is integrally connected to the body.

4. The device of claim 1, wherein the biocompatible material comprises polytetrafluroethylene.

5. The device of claim 1, wherein the body further comprises at least one therapeutic agent.

6. The device of claim 1, wherein the body has a depth of from about 2 mm to about 8 mm.

7. The device of claim 1, wherein the body has a length of from about 20 cm to about 50 cm.

8. The device of claim 1, wherein the edges comprise ridges, bumps, or combinations thereof.

9. The device of claim 1, wherein the body further comprises a reinforcing material.

10. The device of claim 9, wherein the reinforcing material is a metal.

11. A method for locating an arteriovenous fistula and occluding an arteriovenous fistula needle puncture site, the method comprising:
providing a device having a body, the body formed from a self-sealing biocompatible material, the body having a semi-curved shape with two edges that extend along the length of the body;
positioning the device so that the body conforms to the contour of a portion of an arteriovenous fistula with the edges generally parallel to the length of the arteriovenous fistula, the body providing a barrier to limit bleeding from the posterior wall of the arteriovenous fistula;
detecting a tactile clue from the edges of the body when focal pressure is applied to the skin above the arteriovenous fistula.

12. The method of claim 11, wherein the body further comprises a base, the base having a width that is greater than the distance between the two edges of the body.

13. The method of claim 11, wherein the device is at least partially positioned using a tunneler.

14. The method of claim 13, wherein the tunneler comprises a tube and a cap, the tube capable of housing the device, the cap capable of being attached to an end of the tube.

15. The method of claim 11, wherein the biocompatible material comprises polytetrafluroethylene.

16. The method of claim 11, wherein the body further comprises at least one therapeutic agent.

17. The method of claim 11, wherein the body has a depth of from about 2 mm to about 8 mm.

18. The device of claim 11, wherein the body has a length of from about 20 cm to about 50 cm.

19. The device of claim 11, wherein the edges comprise ridges, bumps, or combinations thereof.

20. The device of claim 11, wherein the body further comprises a reinforcing material.
